(19) United States
(12) Patent Application Publication
(10) Pub. No.: US 2008/0027403 A1
(43) Pub. Date: Jan. 31, 2008

(54) Absorbent articles and wipes

(76) Inventors: Rosa Alejandra Hernandez, Cincinnati, OH (US); Ilonka Bibiana Laviz, Mason, OH (US); Irene Spalazzi, Mason, OH (US); Ronald Bosman Visscher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/495,280

(22) Filed: Jul. 28, 2006

(51) Int. Cl. A61F 13/15

(52) U.S. Cl. 604/385.06; 604/385.02

(57) ABSTRACT

An absorbent article combination comprising an absorbent article and a wipe. The absorbent article and/or wipe can include one or more coordinated features. The wipe can be individually packaged and attached to an individually packaged absorbent article.

### FIELD OF THE INVENTION

The present invention relates generally to the field of absorbent articles and wipes.

### BACKGROUND OF THE INVENTION

Absorbent articles, such as sanitary napkins, liners, and tampons, are generally used by women to absorb menstrual or other body exudates. Diapers and incontinence articles are used by babies or other incontinent individuals to absorb and contain body exudates, such as, e.g., urine and/or feces. Often, the use and changing of these articles is messy and unhygienic. The hands or body of the person changing the article can become contaminated due to a failure of the article to contain the exudates or due to exudates that reside on the user's body or garments after removal of the article. In addition, it can be desirable to clean portions of the user's body or garments, which can also lead to contamination of the hands of the person changing the article. Thus, it would be beneficial to have a means for cleaning soiled skin and/or garments while using or changing an absorbent article.

One means for cleaning soiled surfaces is a wipe. Such wipes can include, for example, wet wipes or dry wipes. Wet wipes used for cleansing are often made of a nonwoven material and can comprise a liquid cleansing solution. Some wipes contain a fragrance and/or a lotion. Typically, such wet wipes are generally uniform in color and rectangular in shape. Multiple wipes are often packaged together in a common package, but wipes can also be individually packaged.

Many consumers do not currently use wet wipes when using or changing an absorbent article. Such consumers may not use a wipe even if provided with the article if the consumer cannot quickly appreciate the benefits of the wipe and how it is to be used. Furthermore, the same wipe may not be as effective with different absorbent articles and/or when used by different users on different occasions.

As such, it would be desirable to provide a combination absorbent article and wipe product that communicates product benefits to a user. It would also be desirable to provide different wipes for use with different absorbent articles, and/or for use by different users on different occasions.

### SUMMARY OF THE INVENTION

An absorbent article combination comprising an absorbent article and a wipe, wherein the absorbent article and the wipe include one or more coordinated features is provided. The one or more coordinated features can be, for example, size, scent, odor control, lotion, texture, thickness, printing, coloration, a brand name, a package, a trade dress, and/or usage instructions. The absorbent article combination can further comprise a pouch joined to the absorbent article, the pouch having the wipe disposed therein. The wipe can be a nonwoven material and/or a wet wipe.

A package comprising an absorbent article and a wipe wherein the absorbent article and the wipe include one or more coordinated features is provided. In addition, an array comprising one or more wipes and one or more absorbent articles, wherein the array comprises a plurality of wipes comprising one or more coordinated features and a plurality of absorbent articles comprising one or more coordinated features is provided.

Also provided is an absorbent article combination comprising an absorbent article, a releasable wrapper wherein the releasable wrapper at least partially surrounds the absorbent article, and a pouch joined to the releasable wrapper, the pouch having a wipe disposed therein, wherein one or more of the absorbent article, releasable wrapper, and/or pouch includes a coordinated feature.

### BRIEF DESCRIPTION OF THE DRAWINGS

FIG. 1 is a cross-sectional side view of one embodiment of the present invention.

FIG. 2 is a perspective view of an alternative embodiment of the present invention.

FIG. 3 is a cross-sectional view of one embodiment of the present invention.

### DETAILED DESCRIPTION OF THE INVENTION

The present invention relates to wipes that can be used in conjunction with an absorbent article. The wipe can be coordinated with an absorbent article, such as, for example, to facilitate user recognition, increase efficacy, and/or improve ease of use. Different wipes can be provided for different absorbent articles, different occasions, and/or different users. For example, an absorbent article such as a sanitary napkin that is designed for maximum protection could be coordinated with a wipe that is larger and wider than a wipe that would coordinate with a sanitary napkin that is designed for light menstrual flow. An absorbent article that is scented and/or patterned could coordinate with a wipe that is scented and/or patterned. In certain embodiments, an incontinence article can have a wipe intended primarily for cleaning urine and/or feces, while a sanitary napkin can have a wipe intended primarily for cleaning menses. In addition, a coordinated combination absorbent article and wipe product can communicate product benefits to a user, such as, e.g., odor control, scent, lotion, absorbency, leakage protection, freshness, and/or cleanliness. In certain embodiments, the coordinated wipe can be individually packaged and the individually packaged wipe can be joined to an absorbent article that can be individually packaged.

The combination of a wipe with an absorbent article can provide one or more of several advantages. For example, the wipe can provide for cleansing, which can provide the wearer with increased comfort, physically and/or emotionally. The use of the wipe can also provide a reduction in the odor and/or perceived odor associated with menstruation or other bodily exudates. In addition, the use of the wipe can provide emotional benefits to the user by providing a feeling of freshness and/or cleanliness.

As used herein, the term “absorbent article” refers to devices that absorb and/or contain a substance, such as, for example, body exudates. A typical absorbent article can be placed against or in proximity to the body of the wearer to absorb and contain various exudates discharged from the body.

“Individually packaged,” as used herein, means that only one item is packaged a particular package.

“Absorbent article combination” as used herein refers to an absorbent article with a wipe joined thereto.

As used herein the term “joined” refers to the condition where a first member or component is attached, affixed, or otherwise physically connected to a second member or component either directly or indirectly. An example of an indirectly joined member or component might be where the first member or component is affixed or connected to an intermediate member or component that in turn is affixed or connected to the second member or component. The joined relationship between the first member or component and the second member or component can be fixedly or releasably.

As used herein the term “fixedly joined” refers to a connection that cannot be unattached without at least partially destroying one of the attached components. As used herein the term “releasably joined” refers to a connection that is meant to be easily released.

As used herein, the term “wipe” refers to a piece of material that can be used to clean a user's skin or a surface. The wipe made of any suitable material, such as, for example, nonwoven material, paper material, woven material, knitted material, tufted material, stitch-bonded material incorporating binding yarns or filaments, or material felted by wet-milling. Nonwoven material as used herein is “a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling.” “ISO 9092:1988, Textiles-Nonwovens-Definition” International Organization for Standardization, 1988. As such, the wipe can be a “nonwoven wipe,” that is, a wipe comprising a nonwoven substrate. The wipe can also be a “paper wipe,” that is, a wipe comprising a paper substrate, such as, for example, a wet-laid substrate like toilet tissue or facial tissue.

The term “wet wipe” as used herein refers to a wipe that includes a substrate that is moistened with any fluid prior to packaging and/or use.

As used herein, the term “coordinated” when used with respect to a wipe and an absorbent article in an absorbent article combination, refers to items that are designed to be used and/or sold together, and/or designed to be compatible, complementary, synergistic, and/or easily recognizable as being designed for use together. Coordinated items can have similar features, such as, for example, size relative to other like articles, texture, printing, coloration, scent, odor control, and/or presence of lotion.

The term “coordinated feature” as used herein, refers to a feature that communicates to the user that a coordinated wipe and absorbent article are to be used in combination with each other. A coordinated feature can include, for example, size, scent, lotion, odor control, texture, thickness, coloration, a brand name, a package, a trade dress, a set of usage instructions, and/or printing, such as, e.g., one or more images.

The term “functional benefit” as used herein, refers to a benefit that the wipe and/or absorbent article provides to a user. A functional benefit can be an actual feature, such as, e.g., a lotion or scent, or a perceived benefit, such as, e.g., a feeling of freshness and/or cleanliness.

As used herein, the term “related” means to have characteristics that are logically associated.

As used herein, the term “complementary” refers to two or more elements that when detected together by a user can produce a desired effect that is different from the effect of using one element individually.

As used herein, the term “synergistic” refers to elements that when detected together by a user produce a reaction in the user that is stronger or more desirable than the reaction created when the user detects the elements individually.

As used herein, the term “extended effect” refers to an effect that lasts for a longer period of time compared to the inclusion of one or more coordinated features in either the wipe or the absorbent article individually.

The term “image” when used with reference to printing includes, for example, any type of mark, figure, picture, identification code, symbol, icon, pattern, or text, such as, e.g., a word, nomenclature, number, sentence, or instruction. An image can also be a line, line segment, curved line, band, arrow, area of coloration, or any other printed indicia having a purpose of providing a signal or guide to the user.

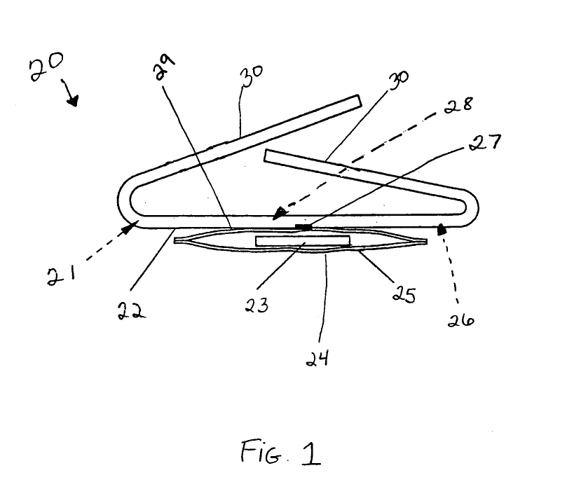

FIG. 1 shows a cross-sectional side view showing an absorbent product combination 20 that comprises a sanitary napkin 21 and an individually packaged wipe 23 that is in a pouch 25. In this embodiment, the pouch 25 is joined to the sanitary napkin 21 by a joining means 27. The pouch 25 can be joined to the sanitary napkin 21 either releasably or fixedly. Any suitable means for joining the pouch 25 to the sanitary napkin 21 can be used. One example of a suitable joining means 27 is an adhesive, such as, e.g., a pressure sensitive adhesive. In certain embodiments, the sanitary napkin 21 and pouch 25 are releasably joined using a glue attachment, such as, for example, hot melt glue. The sanitary napkin 21 can have a releasable wrapper 22 that is either releasably joined to or not joined to the sanitary napkin 21. The releasable wrapper 22 can either partially or completely surround the sanitary napkin 21. In certain embodiments, the pouch 25 can be releasably joined to the releasable wrapper 22 of the sanitary napkin 21. The pouch can also, or alternatively, be fixedly joined to the sanitary napkin 21.

The sanitary napkin 21 shown in FIG. I has a body-facing side 28 and an opposed garment-facing side 26. The pouch 25 has an outwardly facing side 24 and an opposed inwardly facing side 29. The inwardly facing side 29 of the pouch 25 can be joined to the garment facing side 26 of the sanitary napkin 21. FIG. 1 shows the pouch 25 joined to the sanitary napkin 21 in the middle of the garment-facing side 26. The pouch 25 can be joined to the sanitary napkin 21 at any suitable location on the sanitary napkin 21, the releasable wrapper 22, or a package enclosing the sanitary napkin 21. In one embodiment, the pouch 25 is joined to the sanitary napkin 21 at one end 30 of the napkin 21. Alternatively, the pouch 25 could be located on the body-facing side 28 of the sanitary napkin 21. These locations can permit the pouch 25 containing the wipe 23 to be removed and used before or without opening the sanitary napkin 21.

In certain embodiments, the sanitary napkin 21 comprises a pair of flaps that each extend from a longitudinal side margin. Suitable sanitary napkins are described in, for example, U.S. Pat. Nos. 5,346,486; 5,009,653; 4,950,264; 4,917,697; 4,687,478; 4,589,876; and 4,285,343. In this embodiment, the flaps of the sanitary napkin 21 are folded over the topsheet in a topsheet facing relationship, and the pouch 25 containing the wipe 23 is releasably affixed to the flaps such that the topsheet facing relationship is maintained. Such an arrangement is described in, for example, U.S. Pat. No. 6,911,022.

Any pouch 25 suitable for holding a wipe 23 can be used. In certain embodiments, the pouch 25 is separate from the absorbent article prior to attachment. For example, a pouch separate from an absorbent article may not comprise a component of the absorbent article. In other embodiments, the releasable wrapper 22 can comprise a flap (or pouch) to contain a wipe 23. A suitable flap (or pouch) is described in, for example, U.S. Pat. Nos. 4,556,146 and 5,569,230. The absorbent article can comprise a releasable paper wrapper 22 that surrounds the absorbent article to form an individual package and the pouch comprises a portion of the releasable paper wrapper 22, such as, for example, the pouches described in, e.g., U.S. Pat. No. 5,569,230.

With respect to the sanitary napkin 21 shown in FIG. 1, the sanitary napkin 21 can be individually packaged, such as, for example, in a flexible poly wrapper. In certain embodiments, the releasable paper wrapper 22 of the sanitary napkin 21 can surround the sanitary napkin 21 to form a package. The individually wrapped sanitary napkins can be packaged in a retail package, such as, for example, a box or bag, such as a poly bag.

Although FIG. 1 shows a sanitary napkin 21 as the absorbent article, any suitable absorbent article can be used, such as, e.g., pantiliners, incontinence articles, interlabial pads, or baby diapers. Suitable pantiliners are disclosed in U.S. Pat. No. 4,738,676. Other absorbent articles are described in, for example, U.S. Pat. Nos. 6,059,764; 5,951,536; 5,824,004; 5,702,382; 5,683,375; 5,658,269; 5,439,458; 5,304161; and 5,300,054.

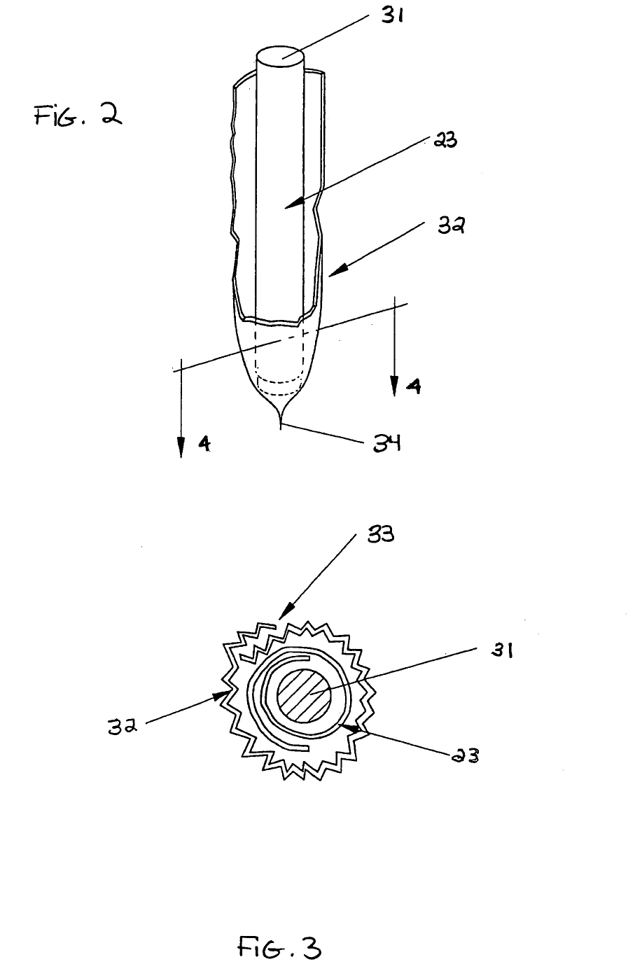

FIGS. 2 and 3 show an example of an embodiment for packaging a wipe 23 with a tampon 31. The tampon 31 can be any known form, such as, for example, tampons disclosed in U.S. Pat. No. 4,475,911. The wipe 23 can be wrapped around the tampon 31. The wrapped tampon 31 can be wrapped with a moisture and vapor impervious overwrap 32. The side 33 and ends (one of which is shown as 34) of the overwrap 32 are sealed. The tampon package can be provided with a releasable seal at the side 33, and/or at least one of the ends 34 for use in opening the package. Any suitable configuration and/or means for joining the tampon 31 and the wipe 23 can be used. For example, in certain embodiments, the wipe 23 can be releasably attached to the overwrap 32 of the tampon 31.

In certain embodiments, the wipe 23 and/or absorbent article can include one or more coordinated features. The one or more coordinated features can communicate to the user that a coordinated wipe 23 and/or absorbent article can be intended to be used in combination with each other, and/or can provide perceived, improved, and/or synergistic results when included in the wipe 23 and/or absorbent article. Thus, the coordinated feature can include, for example, size, scent, odor control, lotion, texture, thickness, printing, such as, e.g., one or more images, coloration, a brand name, a package, a trade dress, and/or a set of usage instructions. In certain embodiments, the wipe 23 and/or absorbent article are “coordinated” in that the wipe 23 and/or absorbent article are designed to be used and/or sold together, and/or designed to be compatible, complementary, synergistic, and/or easily recognizable as being designed for use together. The wipe 23 can include one or more coordinated features that are the same or different as one or more coordinated feature included in the absorbent article. A wipe 23 and/or absorbent article that include different coordinated features can be considered “coordinated” if the combination of coordinated features indicates to a user that the wipe 23 and/or absorbent article are designed to be used and/or sold together, and/or designed to be compatible, complementary, synergistic, and/or easily recognizable as being designed for use together

In certain embodiments, the wipe 23 and/or absorbent article can include a coordinated feature that can be size. For example, a wipe 23 intended for use with a larger absorbent article, such as, e.g., a sanitary napkin 21 and/or tampon 31 intended for maximum protection, can be larger and/or wider than a wipe 23 intended to be used with a regular sized absorbent article. In addition or alternatively, a wipe 23 intended for use with a slender, junior, and/or smaller sized absorbent article can be smaller and/or narrower than a wipe 23 intended to be used with a super, overnight, and/or maximum protection absorbent article.

In certain embodiments, the wipe 23 and/or absorbent article can include a coordinated feature that can be one or more scents. For example, a wipe 23 intended for use with a scented absorbent article can include one or more scents that is the same as, related to, complimentary to, and/or synergistic with the one or more scents of the absorbent article. The inclusion of the coordinated feature, such as, e.g., scent, can provide an extended effect.

In certain embodiments, the wipe 23 and/or absorbent article can include a coordinated feature that can be odor control. For example, a wipe 23 intended for use with an absorbent article including odor control can include odor control that is the same as, related to, complimentary to, and/or synergistic with the odor control of the absorbent article. The inclusion of the coordinated feature, such as, e.g., odor control, can provide an extended effect.

In certain embodiments, the wipe 23 and/or absorbent article can include a coordinated feature that can be texture and/or thickness. For example, a wipe 23 intended for use with a thicker and/or textured absorbent article can be thicker and/or textured. The absorbent article can be a thick sanitary napkin 21 that can provide users with a cushiony look and feel and/or the coordinated wipe 23 can be thick and cushiony to indicate to the user that the sanitary napkin 21 and wipe 23 are intended to be used together.

In certain embodiments, the wipe 23 and/or absorbent article can include a coordinated feature that can be one or more lotions. For example, a wipe 23 intended for use with an absorbent article that includes one or more lotions can also include one or more lotions that provide an extended effect and/or one or more benefits that are the same, related to, complementary to, and/or synergistic with the one or more benefits provided by the one or more lotions included in the absorbent article. The inclusion of the coordinated feature, such as, e.g., lotion, can provide an extended effect.

In certain embodiments, the wipe 23 and/or absorbent article can include a coordinated feature that can be printing and/or coloration. For example, a wipe 23 intended for use with an absorbent article that has a printing and/or coloration can have a printing or coloration that is the same as, related to, complementary to, and/or synergistic with the coloration of the absorbent article. The wipe 23, absorbent article, and/or exterior packaging can each be printed with the same design and/or have printing or coloration in the same shade. In such an arrangement, the printing may be disposed on, for example, the wipe 23, the pouch 25, the absorbent article, the releasable wrapper 22, a package containing the absorbent article, and/or an exterior packaging such as, for example, a bag or a box in which a plurality of wipes, absorbent articles, and/or absorbent article combinations are packaged. In certain embodiments, the wipe 23, pouch 25, absorbent article, and/or package can be printed with information about the corresponding absorbent article and/or wipe 23. For example, a wipe 23 packaged with a liner that is printed with purple flowers could also be printed with purple flowers. Alternatively, a wipe 23 packaged with a liner that is printed with purple flowers could be printed with other images or text in a shade similar to the purple used on the liner.

In addition, or alternatively, the absorbent article can have one or more coordinated features that are one or more functional benefits, such as, for example, increased absorbency, leakage protection, dryness, scent, odor control, and/or lotion, and the coordinated wipe 23 can have one or more coordinated features that are printing, embossment, and/or coloration that communicate to the user the one or more functional benefits of the absorbent article. For example, the absorbent article can have one or more coordinated features that can be one or more functional benefits such as, for example, an apertured topsheet, and the wipe 23 can have one or more coordinated features that is printing and/or coloration, such as, e.g., holes and/or slits, to communicate to the user the one or more functional benefits of the absorbent article. The absorbent article could also, or alternatively, have one or more coordinated features that can be one or more functional benefits that can be scent and/or odor control, and the wipe 23 can have one or more coordinated features, such as, e.g., printing and/or coloration, such as, for example, flowers, to communicate to the user the one or more functional benefits of the absorbent article.

The wipe 23 can be printed with any suitable image and/or in any suitable color. The colors of printed regions can be uniform, and/or they can be varying shades or hues of one color, and/or they can be different colors. The wipe 23 can be printed using any printing technology suitable to add color and in any suitable manner, such as, for example, discrete or continuous printing. The wipe 23 can be printed in any suitable location using any suitable ink. In addition, or alternatively, the wipe 23 can be embossed. At least one or all of the embossed depressions can have therein a substance such as ink to provide visible color. One or more fibers of the substrate of the wipe 23 can also, or alternatively, include one or more colorations.

The coordinated feature can be included in any aspect of the wipe 23 and/or absorbent article. For example, in the case of a wipe 23, the coordinated feature can be included in the substrate, such as, for example, by printing, embossing, coloration, and/or the addition of a lotion, scent, and/or cleaning composition to the substrate; the pouch 25; and/or any external packaging, such as, e.g., a box or a bag. In the case of an absorbent article, the coordinated feature can be included in the absorbent article, such as, for example, the topsheet, absorbent core, backsheet, release paper, and/or releasable paper wrapper, by, e.g., printing, embossing, coloration, and/or the addition of a lotion, scent, or other composition to the absorbent article; any individual packaging; and/or any external packaging, such as, for example, a box or a bag.

In certain embodiments, the coordinated feature can be a brand name. A brand name can increase user recognition and/or can communicate to the user about the nature of the product. Furthermore, a brand name can indicate to the user that the wipe 23 and absorbent article are intended to be used together, and can alert a user to potential related, complementary, synergistic, and or extended benefits that can occur with use of the wipe 23 with the coordinated absorbent article.

The coordinated element can also, or alternatively, be a package, such as, for example, a release paper wrapper, a pouch 25 containing a wipe 23, and/or an external package, which can have related, similar, and/or identical images and/or colorations. As with the brand name above, a package for a wipe 23 and/or absorbent article having related, similar and/or identical images and/or coloration can easily and recognizably indicate to a user that the wipe 23 and/or absorbent article are intended to be used together, such as, for example, to provide synergistic results.

The coordinated feature can be a trade dress that indicates to the user that the wipe 23 and/or absorbent article can be used in combination with each other. As used herein, the term “trade dress” indicates the appearance and image of the wipe 23 and/or absorbent article. Trade dress can be a combination of physical features such as the size, texture, shape, color and color combinations, graphics, packaging material, and/or other physical features.

The coordinated feature can be a set of usage instructions which are attached to the absorbent article combination and/or the wipe 23 and/or absorbent article therein. The set of usage instructions may be provided in virtually any location and in any form (e.g., visual, audio, tactile such as braile, etc.), as long as it is perceivable to a user purchasing and/or using the absorbent article combination. Thus, the set of usage instructions may be provided with the absorbent article combination, and/or on a location such as a pamphlet, a computer screen, a printed ticket, a kiosk, a sign, a product package, an advertisement, a product display, an Internet website, a video, and/or a combination thereof.

In certain embodiments, an absorbent article combination can be personalized for the user. For example, a user profile may be used and/or cross-referenced with a mathematical algorithm, a user profiling database, a statistical analysis, etc. to determine the user's typical or expected usage of the absorbent article combination. This user profile, in turn, may be used to provide the user with a personalized combination containing the appropriate products. In certain embodiments, the products in the personalized combination can comprise a coordinated feature.

In certain embodiments, the wipe 23 can be individually packaged and can be joined to the absorbent article. One individually packaged wipe 23 can be joined to one individually packaged absorbent article, more than one individually packaged wipe 23 can be joined to one individually packaged absorbent article, one individually packaged wipe 23 can be joined to more than one absorbent article that can be individually packaged, more than one wipe packaged together can be joined to one or more individually packaged absorbent articles, one or more individually packaged wipes can be joined to one or more absorbent articles packaged together, or any suitable combination. In certain embodiments, the wipe can be individually packaged, but is not joined to the absorbent article. For example, the wipes can be packaged in individual pouches and a plurality of individual pouches containing wipes can be packaged with absorbent articles in the same exterior packaging or the plurality of individual pouches can be packaged in a different exterior package from that of the absorbent article. The wipes and the absorbent articles can be co-marketed, such as, e.g., designed to be sold and/or used together, such as, for example, for the same purpose. Alternatively, a plurality of wipes can be packaged together in a common package and then packaged with absorbent articles in the same exterior packaging or the plurality of wipes can be packaged together in a common package that is different from that of the absorbent article. In certain embodiments, a plurality of individually packaged wipes are packaged with a plurality of individually packaged absorbent articles wherein the individual package of one or both of the absorbent article or the wipe is provided with an attachment means that allows attachment of the wipe 23 to the absorbent article, such as described in, for example, WO 2005/115295.

In certain embodiments, a plurality of wipes is packaged together. The wipes can be packaged in individual pouches and then packaged in any suitable packaging. Alternatively, the wipes can be packaged together, such as, for example, in a stack, and then packaged in packaging, such as, for example, a hard-sided container or tub; or a soft-sided package such as a pouch or bag.

In certain embodiments, an array comprising one or more wipes and one or more absorbent articles is provided. The array can include wipes including one or more coordinated features and absorbent articles including one or more coordinated features. The one or more coordinated features of the wipes can be the same or different as the one or more coordinated features of the absorbent articles. In certain embodiments, the one or more coordinated features of the wipes can be related to the one or more coordinated features of the absorbent articles. The array can comprise a plurality of wipes including a first coordinated feature and a plurality of absorbent articles including a first coordinated feature; and a plurality of wipes including a first coordinated feature and a plurality of absorbent articles including a first coordinated feature. For example, an array of sanitary napkins could include sanitary napkins of different sizes, such as, e.g., regular and super, and wipes of a first size to coordinate with the regular sanitary napkins and wipes of a second size to coordinate with the super sanitary napkins. In certain embodiments, each wipe 23 in the array is individually packaged and attached to an individually packaged absorbent article. In certain embodiments, more than one wipe 23 can be attached to a single absorbent article or, alternatively, one wipe 23 can be attached to more than one absorbent article.

The wipe 23 can be a nonwoven wipe 23 comprising a substrate comprising a nonwoven web of natural fibers, synthetic fibers, or mixtures of natural and synthetic fibers wherein the substrate is not considered paper. Suitable natural fibers include, e.g., cellulosic fibers, such as wood pulp fibers, cotton, and rayon. Suitable synthetic fibers include fibers commonly used in textiles, such as, for example, polyolefins, such as polyester and polypropylene fibers. In certain embodiments, the wipe 23 is a nonwoven wipe comprising a substrate that comprises viscose rayon and/or wood pulp. Alternatively, the wipe 23 can be a paper wipe, such as, e.g., a wipe 23 comprising a substrate that is made of a wet-laid material in a manner similar to toilet tissue or facial tissue. In certain embodiments, the wipe can comprise silk.

The wipe 23 can be “flushable”, that is, it is of a size of is capable of disintegrating to pieces sufficiently small such that when flushed in a toilet and when being transported in the sewer system, it does not plug any element of a sewer system. Any wrapping material associated with the wipe 23, such as, for example, the pouch 25material, can also be flushable. Wrapping materials that meet such criteria can include, for example, water soluble materials, such as, e.g., silicone-treated polyvinyl alcohol films, films coated with a polyvinyl alcohol, and tissue coated or impregnated with polyvinyl alcohol. Suitable materials are disclosed in, e.g., U.S. Pat. No. 4,755,421.

The wipe 23 can be any size suitable for cleansing the skin or providing other benefits when using or changing an absorbent article. In certain embodiments, the wipe 23 can be rectangular. The wipe 23 can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size.

The wipe 23 can be a wet wipe that comprises a liquid-based cleansing solution. The wipe 23 can also be scented. In certain embodiments, the wipe 23 can comprise a nonwoven fabric impregnated with the cleansing solution. The cleansing solution can include, for example, surfactants, alcohols, fragrance compositions, antimicrobial agents, pH buffers, and combinations thereof. The wipe 23 can also contain substances such as silicones that can inhibit body exudates from adhering to the user's body. The cleaning solution can also contain skin conditioning substances similar to those used in hand lotions, such as, e.g., aloe and Vitamin E; other substances for applying to the skin, such as, e.g., witch hazel; or any other substances known in the art for inclusion in wet wipes.

The wipe 23 can comprise a lotion, such as, for example, an aqueous or non-aqueous lotion. In certain embodiments, the lotion can increase the cleaning capabilities of the wipe 23 by aiding removal of soiling from the skin. The lotion can also provide skin benefits by reducing friction between the skin and the wipe 23 and/or by containing materials that can soothe the skin, such as, for example, aloe or chamomile. Examples for suitable lotions are disclosed in, for example, EP 808151; EP 763,341, and WO 00/57843.

One or more fragrance compositions can be included in the cleansing solution in order to produce a scented wipe 23. Alternatively, the one or more fragrance compositions can be included in the wipe 23 separately from any cleansing solution such as, for example, by applying the fragrance composition to the wipe 23 prior to or after any cleansing solution may be added. Another example might be a wipe 23 that includes a separate component such as, for example, a carrier material comprising a fragrance composition. Additionally, the one or more fragrance compositions may be disposed together or separately on at least a portion of the wipe 23.

Other embodiments can include dry wipes or wipes containing non-aqueous cleaning solutions such as, for example, mineral oils, and the emollient described in U.S. Pat. No. 4,481,243.

In certain embodiments, the wipe 23 can be packaged in a pouch. Any suitable pouch 25 for holding a wipe 23 can be used. For example, the pouch 25 can be made of paper and/or other suitable materials, such as, e.g., polyethylene. When the wipe 23 is a wet wipe, any pouch 25 suitable for maintaining the wetness of the wipe 23 can be used. For example, the pouch 25 can comprise at least one barrier material suitable for preventing evaporation of moisture from the wipe 23 can be used. Suitable barrier materials include, for example, laminates with a layer of ethylene vinyl alcohol (EVOH), propylene vinyl alcohol (PVOH), or aluminum sandwiched between polymeric film layers, such as LDPE (low density polyethylene); metallized, such as aluminized plastic films; metal foils; oriented polyethylene terephtalate (PET); PETG (glycol-modified PET); oriented polyamide; aromatic polyamide; or polymeric films like polyethylene films. In certain embodiments, the pouch 25 comprises paper and/or foil. The pouch 25 can also, or alternatively, comprise an inner layer comprising polyethylene (PE), an outer layer comprising a copolymer of polyethylene (PE) and polypropylene (PP) and a subjacent intermediate layer comprising polypropylene (PP).

In certain embodiments, one or more sides of the pouch 25 can be sealed to prevent substantial evaporation of the liquid solution from the time the product is manufactured to the time it ;is used by the user. Any number of sealing mechanisms may be used including, for example, heat and/or pressure seals, ultrasonic seals, glue seals, zippered track sealing systems such as Dow Company's “ZIP LOCKS” or combinations thereof. In certain embodiments, the pouch 25 can be hermetically sealed. In addition, the pouch 25 may be lined with foil, or other suitable material to further reduce evaporation.

The pouch 25 may include a means for unsealing (or opening) the pouch 25 in order to access a wipe 23 contained therein. For example, the pouch 25 can have a scored or die cut line of weakness to allow the pouch 25 to be opened easily with a tab, easy peel label or any other opening mechanism; a “Dry-edge” sticker mechanism, such as, e.g., a sticker with a dry edge for grasping; releasable adhesives; releasable heat and/or pressure seals; perforations; rupture seals; tear seals; or the pouch 25 can be made of a relatively easy to tear material such as, for example, an easy to tear film.

Any suitable joining means can be used to attach the pouch 25 and absorbent article of the absorbent article combination. In certain embodiments, the joining means on the outside of the package of the wipe 23 can be selected and applied such that a sufficient degree of joining between the package of the absorbent article and the package of the wipe 23 is generated. Any suitable adhesive can be used to join the absorbent article and the wipe 23, such as, for example, a pressure-sensitive adhesive, hot melt adhesive, cold melt adhesive, or solvent-based adhesives. The adhesive can be applied to the absorbent article and/or wipe 23 as a continuously coated area or as a pattern, such as, e.g., dots, stripes, spirals, or beads. In certain embodiments, the absorbent article and the wipe 23 can be joined by one or more mechanical fasteners. Suitable mechanical fasteners include, for example, hook and loop fasteners.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and, modifications that are within the scope of this invention.

What is claimed is:

1. An absorbent article combination comprising:
a. an absorbent article; and
b. a wipe,
wherein the absorbent article and the wipe include one or more coordinated features selected from the group consisting of: size, scent, odor control, lotion, texture, thickness, printing, and coloration.

2. The absorbent article combination of claim 1, wherein the one or more coordinated features are a brand name, a package, a trade dress, and usage instructions.

3. The absorbent article combination of claim 1, wherein at least one coordinated feature of the absorbent article is the same as at least one coordinated feature of the wipe.

4. The absorbent article combination of claim 1, wherein the absorbent article includes one or more functional benefits, and wherein one or more coordinated features of the wipe is printing and/or coloration.

5. The absorbent article combination of claim 1, wherein the combination further includes one or more elements selected from the group consisting of: a release paper, a releasable paper wrapper, a pouch, and/or an external package, wherein the one or more elements include one or more coordinated features.

6. The absorbent article combination of claim 1, wherein the combination further comprises a pouch joined to the absorbent article, the pouch having the wipe disposed therein.

7. The absorbent article combination of claim 1, wherein the wipe comprises a substrate that comprises a nonwoven material.

8. The absorbent article combination of claim 1, wherein the wipe is a wet wipe.

9. A package comprising:
a. an absorbent article; and
b. a wipe,
wherein the absorbent article and the wipe include one or more coordinated features selected from the group consisting of: size, scent, odor control, lotion, texture, thickness, printing, and coloration, and
wherein one or more coordinated features of the absorbent article is the same as one or more coordinated features of the wipe.

10. The package of claim 9, wherein the one or more coordinated features are a brand name, a package, a trade dress, and/or usage instructions.

11. The package of claim 9, wherein the wipe is individually packaged.

12. The package of claim 9, wherein the wipe is a wet wipe.

13. An array comprising one or more wipes and one or more absorbent articles, wherein the array comprises a plurality of wipes comprising one or more coordinated features and a plurality of absorbent articles comprising one or more coordinated features the one or more coordinated features selected from the group consisting of size, scent, odor control, lotion, texture, thickness, printing, and coloration.

14. The array of claim 13, wherein the one or more coordinated features are a brand name, a package, a trade dress, and/or usage instructions.

15. The array of claim 13, wherein each wipe in the array is individually packaged in a pouch, wherein the pouch is attached to one or more individually packaged absorbent articles.

16. An absorbent article combination comprising:
a. an absorbent article;
b. a releasable wrapper wherein the releasable wrapper at least partially surrounds the absorbent article; and
c. a pouch joined to the releasable wrapper, the pouch having a wipe disposed therein,
wherein two or more of the absorbent article, releasable wrapper, or pouch includes a coordinated feature slected from the group consisting of: size, scent, odor control, lotion, texture, thickness, printing, and coloration.

17. The absorbent article combination of claim 16, wherein the coordinated feature is a brand name, a package, a trade dress, and/or usage instructions.

18. The absorbent article combination of claim 16, wherein the absorbent article is a sanitary napkin.

19. The absorbent article combination of claim 16, wherein the wipe comprises a substrate that comprises a nonwoven material.

20. The absorbent article combination of claim 16, wherein the wipe is a wet wipe.
